(19) United States
(12) Patent Application Publication
(10) Pub. No.: US 2006/0079838 A1
(43) Pub. Date: Apr. 13, 2006

(54) Movable Balloon anchor for medical devices

(76) Inventors: Steven C. Walker, Waxahachie, TX (US); Pamela A. Howard, Allentown, PA (US); Eben Howard, Allentown, PA (US)

Correspondence Address: CAHN & SAMUELS LLP, 2000 P STREET NW, SUITE 200, WASHINGTON, DC, 20036, US

(21) Appl. No.: 10/959,991

(22) Filed: Oct. 8, 2004

(51) Int. Cl. A61M 29/00

(52) U.S. Cl. 604/104; 604/175; 604/101.05

(57) ABSTRACT

This invention relates to inflatable anchor members for securing devices in an orifice. Anchor member comprises a fluid tight material having an inflatable interior. The anchor is configured to include an opening for receiving a device. The anchor may be inflated to secure the device within its opening. The anchor includes a fill line for receiving and removing fill material from the anchor. The anchor may be disposed inside the chest of a patient and inflated to secure a chest tube in position without the need for sutures. A second inflatable anchor may be used and positioned on an opposite side of an orifice from the first anchor to hinder movement of a device passing therethrough.

### I. FIELD OF THE INVENTION

The present invention relates to novel medical insertion devices and novel means for securing said devices during treatment of a patient. More particularly, the present invention relates to novel chest tubes, intravenous lines, central lines, and peritoneal catheter devices and novel methods and means for securing these devices when treating a patient.

### II. BACKGROUND OF THE INVENTION

There are numerous situations in which it is desirable or necessary to secure a medical instrument in place in an incision, orifice or wound of a patient. This is true whether treating humans or other animals. For example, as will be appreciated, a particular need for securing a medical device arises in the event of chest trauma. In the United States, chest injuries alone are responsible for one-fourth of all trauma deaths. On the battlefield, the death rate from chest wounds is even higher. Many chest trauma casualties could be prevented through early recognition of the injury followed by prompt management. In battlefield and mass casualty situations, the ability to quickly treat multiple patients is critical.

The lungs are surrounded by a pleural sac that consists of two membranes—the visceral pleurae and the parietal pleurae. The parietal pleura lines the thoracic wall, and the visceral pleura surrounds the lung. The pleural space is the space between these two layers of pleurae and contains a thin layer of serous pleural fluid that provides lubrication for the pleurae and allows the layers of pleurae to smoothly slide over each other during respiration events.

Pneumothorax is the medical condition resulting from air entering the pleural space. Hemothorax is the medical condition resulting from blood entering the pleural space. Both of these conditions can result from an injury or trauma to the chest. More importantly, pneumothorax and hemothorax are potentially lethal unless treated promptly. Common causes of pneumothorax and hemothorax include penetrating injuries (e.g., gunshot and stab wounds or injuries occurring as the result of a surgical procedure) and blunt injuries (e.g., from direct blows, crushing injuries, blasts, or falls). Pneumothorax may also occur as a result of the use of positive end-expiratory pressure (PEEP) in connection with mechanical ventilation procedures, or spontaneously as a result of emphysematous blebs (air spaces that may occur in the lung as a result of emphysema).

Normally, the pressure in the pleural space is much lower than the atmospheric pressure. Following trauma, air may enter the pleural space in several ways, e.g., through a communication between the pleural space and the outside air, or a leak from disrupted alveoli, bronchi or ruptured esophagus. The entry of air into the pleural space (pneuomthorax) results in an increase in the pressure in the pleural space. The increase of pressure in the pleural space compresses the lung, which can cause a potentially fatal condition known as a collapsed lung.

Eliminating pneumothorax requires prompt decompression of the pleural space, usually accomplished by the insertion of a chest tube and evacuation of the air. Similar procedures are followed during the occurrence of a hemothorax to remove blood from the pleural space. More specifically, in order to decompress the pleural cavity, a chest tube is inserted through the appropriate intercostal space, which is the area between adjacent ribs. Typically the intercostal space is approximately 1-2 cm in size. However, there are significant individual differences depending on the size of the individual, and the phase of the respiratory cycle (the intercostal spaces widen during normal inspiration). Furthermore, there are substantial regional size differences, e.g., the intercostal spaces are deeper anteriorly than posteriorly, and deeper between the superior than the inferior ribs. The lateral part of the intercostal space is the widest zone of the intercostal space (i.e., at the anterior axillary line). In addition to the differences in size from one individual to the next, the composition of the chest wall itself can vary from person to person and also differs based on the gender of the patient. The male chest wall is composed of a greater percentage of muscle tissue than the female chest wall. On the other hand, the female chest wall is composed of a greater percentage of adipose tissue than the male chest wall. Each intercostal space contains three muscles: the innermost intercostal muscles, the internal intercostal muscles, and the external intercostal muscles. In addition, each intercostal space contains a neurovascular bundle (intercostal vein, artery and nerve) that runs below the ribs. Further, the chest wall is covered superficially by muscles, connective tissue and skin. For example, the chest wall, in the fifth intercostal space, anterior axillary line is covered externally by the serratus anterior muscle. The chest wall thickness (CWT) is defined as the length from the thoracic epidermal surface to the parietal pleural lining of the lung. As with the intercostal spaces and chest wall composition, there can be a great variation in chest wall thickness from individual to individual and from location to location in the same individual. Further, the position of the patient can also affect the CWT; the CWT is a few millimeters less when the patient is in a reclined position (torso 45 degrees from horizontal) as compared with the same measurement taken when the patient is in the supine position.

The above-described physical differences between individuals must be considered when inserting a chest tube into a patient. There are several other key factors that come into play when inserting chest tubes, including insertion location, penetration angle, and depth. The primary goals of the tube insertion are to effectively evacuate the unwanted air/blood from the pleural space while also avoiding or minimizing injury to the intercostal neurovascular bundle, lungs and other internal structures. In addition, the chest tube must be well secured to the chest wall so that it cannot be accidently dislodged, and it must also be easily removable once the pneumo/hemothorax is absorbed.

Several techniques are currently used to insert and secure a chest tube in place. Each of these prior art techniques typically involve relatively lengthy manual procedures that require sutures to secure the chest tube to the chest wall. The most common technique for inserting and securing chest tubes involves surgical preparation and draping at the site of the tube insertion (usually at the nipple level-fifth intercostal space, anterior to the midaxillary line on the affected side), administering of local anesthesia to the insertion site, and making a 2-3 cm horizontal incision. A clamp is inserted through the incision and spread until a tract large enough to accept a finger is created. Next, the parietal pleura is punctured with the tip of a clamp, and the physician places a gloved finger into the incision to clear adhesions and to confirm the presence of a free pleural space locally. The proximal end of the chest tube is clamped and the tube is advanced into the pleural space. As the chest tube is inserted, it is directed posteriorly and superiorly. In this position, the chest tube will effectively clear the pleural space of both air and blood.

Once the chest tube is appropriately in place (determined by listening to air movement using a stethoscope), the tube is preferably connected to a one-way valve, such as a Heimlich valve, in order to clear air/blood from the pleural space. The tube must then be sutured to the skin to prevent its movement from the desired location. A dressing is then applied and the tube is taped to the chest.

Insertion and securing a chest tube using this standard technique can require more than 15 minutes to accomplish by a physician and requires extensive medical training to be performed properly. Further, while performing the procedure, the physician must attend to the patient receiving the chest tube and thus is precluded from attending to other patients, even in mass casualty situations. The need to suture the chest tube in place slows the process dramatically.

Various other specialized techniques are known in the art for inserting a chest tube, including the use of a rigid trocar (a sharp-pointed instrument equipped with a cannula); “over-the-wire” techniques (involving the insertion of a needle, attached to a syringe, through an incision and into the pleural cavity, and the introduction of a guide wire used to guide the insertion of progressively larger dilators or angioplasty balloons, and finally a chest tube); peel-away introducers for the insertion of mini-thoracostomy tubes in patients with spontaneous pneumothorax; and disposable laparoscopic trocar-cannulae.

However, most of these techniques also require that an incision be made to initiate the insertion. As will be appreciated by one of ordinary skill in the art, an incision reduces the “snugness” of the device with respect to the chest wall. An incision therefore reduces the stability of the device which may cause the device to move, change the angle of penetration or result in an accidental disengagement of the device from the chest wall. Migration of a chest tube from its proper location is a particular problem experienced in the art. Even when the time is spent to suture a chest tube in place, the tube may migrate or be easily, albeit accidentally, pulled out. If the tube gets pulled out far enough another tube may need to be placed and the patient can experience complications such as a reaccumulation of air or fluid in the chest cavity. Also, if the tube is simply reinserted after migrating out, the patient may develop empyema from the bacteria resulting from an unsanitized tube being inserted into the incision.

Moreover, in some of these techniques, pointed instruments remain inserted in the patient during use. The sharp tips of these devices lie in the vicinity of internal organs, thereby increasing the possibility of injury resulting from these procedures or any migration of the device. These situations underscore the importance of having a reliable means of securing the instrument in the desired location and hindering migration. These drawbacks are exacerbated in situations where there are mass casualties, the procedures must be performed under field conditions, and/or where movement of the patient becomes necessary.

Accordingly, there remains a long-felt, yet unresolved, need in the art for a quick and reliable method of securing medical devices. Likewise, there remains a need in the art for a quick and reliable means of securing and removing a medical device from a patient. There also remains a need for a non-suture means of securing medical devices in place and an improved method of preventing migration of medical devices.

### III. SUMMARY OF THE INVENTION

The present invention overcomes the serious practical problems described above and offers new advantages as well. One object of the invention is to provide a means for securing a medical instrument in an incision, orifice or wound of a patient. According to this object of the invention, one aspect of the invention is to provide a quick and reliable means of securing a medical device in place. According to this object, another aspect of the invention is to provide a means which also allow quick retraction of the device from the patient. According to these aspects of the invention, one advantageous feature of the invention is the provision of means for removably securing a medical device in place without the need for sutures.

Another object of the invention is to provide a medical device including a quick a reliable means for securing the device in a desired position. According to this object of the invention, one aspect of the invention is to provide a device with an anchoring means for securing the device in place. According to this object, another aspect of the invention is to provide the device with an anchoring means which can be quickly released to provide for quick retraction of the device. According to these aspects of the invention, one advantageous feature of the invention is the provision of an anchoring means which may be used with or without sutures to secure the device in place.

These and other objects, aspects and features of the invention may be realized by the provision of an inflatable anchoring member. The inflatable anchoring member may have any suitable configuration adapted to allow the member to serve as an anchor to hold a device in place. In one embodiment, the anchor comprises an annular member that may be inflated to constrict the circumference of a device passing therethrough. The annular member is preferably inserted inside an incision, orifice or the like, in a deflated state and then inflated to a size which prevents the anchor from exiting the area. The constriction of the anchor upon inflation hinders the device's migration.

According to one advantageous feature of the invention, the anchor member may be provided with a means for inflating and deflating the anchor member. In one embodiment, the anchor includes a fill line having a fluid tight fill port. The fill port receives the tip of a syringe or like device to allow a fluid, such as water, saline or air, to be communicated to the anchor. Preferably, the fill port is adapted to remain fluid tight upon retraction of the fluid supply syringe, such as by use of a spring valve. The spring valve is actuated by the tip of the syringe to allow fluid to be supplied or removed from the anchor's interior. Alternatively, the fluid line can be supplied with a fill fluid by any suitable means as long as there is a way to hinder the anchor from prematurely deflating. In yet another embodiment, the anchor may comprise two fillable bodies, wherein a first body is disposed on one side of an opening and the second body is disposed on the other side of the opening. This configuration provides a sandwiching anchor aspect to the invention. Each of the anchor bodies may be selectively inflated and deflated via their own fill lines. Alternatively, both bodies are in communication with a common fill line having a fill port for receiving fill material.

According to the invention, the anchor(s) preferably provides for the removal of the inflation fluid or fill material via the fill line or other suitable means, such as aspiration with a syringe or the like.

According to another advantageous feature of the invention, the anchor member is configured to be moveable along a length of the device's body. In a preferred embodiment, the anchor is configured such that it can be positioned along the length of the body of the device, whereby upon inflation it holds the device at a desired depth. More preferably, the anchor is configured to also allow the device to be held in a desired orientation.

It is another object of the present invention to provide devices having inflatable anchors. It is also an object of the invention to provide devices with deflatable anchors. It is another object of the present invention to provide devices having an adjustable anchor. These and other objects, aspects and advantageous features of the invention may be realized by providing a device having an inflatable and deflatable anchor integral therewith.

According to one advantageous feature of the invention, there is provided a device incorporating the anchor member(s) and means described above. According to another advantageous feature of the invention, there is provided a chest tube incorporating an inflatable anchor as described above.

It is yet another object of the invention to provide methods and means for using the anchors and devices of the present invention described above. A presently preferred method for using a chest tube embodiment of the invention includes the steps of moving an annular anchor member to a predetermined position on the body of the chest tube, inserting the chest tube to a predetermined depth such that said tube is inside the chest wall, and filling the anchor with a fluid from a syringe from a fill line associated with said anchor. In another preferred embodiment, a first anchor body is disposed inside the chest cavity and filled with a fluid and a second anchor body is positioned outside the chest wall and filled with a fluid.

Given the following enabling description of the drawings, the apparatus should become evident to a person of ordinary skill in the art.

### IV. BRIEF DESCRIPTION OF THE DRAWINGS

The present invention is described with reference to the accompanying drawings. In the drawings, like reference numbers indicate identical or functionally similar elements.

FIG. 1 illustrates an embodiment of a chest tube device including an inflatable anchor according to the invention.

FIG. 2 depicts an embodiment of a chest tube after deploying the anchor in the inside chest wall of a patient.

FIG. 3 depicts an embodiment of a chest tube after deploying a first anchor body inside the chest wall of a patient and a second anchor body outside the chest wall of the patient.

### V. DETAILED DESCRIPTION OF THE DRAWINGS

The present invention is based, in part, on the discovery that alternatively inflatable and deflatable members associated with a device for insertion in an orifice could constrict the movement of the device until desired. While the present invention will be described in connection with a chest tube 10 having an annular balloon anchor, it will be readily apparent to one of ordinary skill in the art that the present invention can be applied to a multiplicity of fields and uses. While preferred, the fields should not be deemed limited to medical devices for veterinary or human treatment. Moreover, while preferred, the uses should not be deemed limited to chest tubes, intravenous lines, central lines or peritoneal catheters.

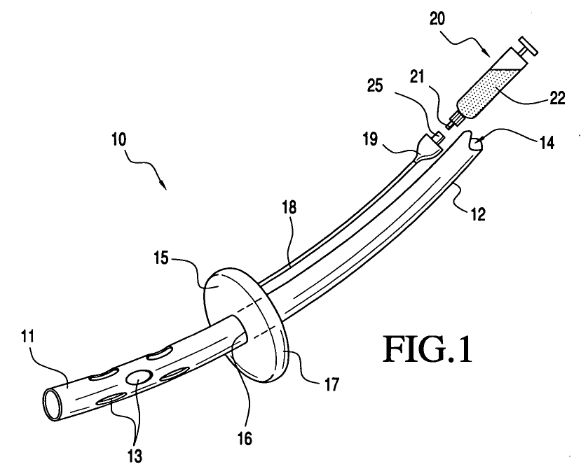

FIG. 1 depicts a presently preferred embodiment of the invention. As shown, chest tube 10 includes insertion end 11 and non-insertion end 12. Insertion end 11 includes chest tube holes 13 providing communication with tube passageway 14. Tube passageway is connected to a one-way valve (not shown) in order to assist clearing air/blood from the pleural space during pneumothorax or hemothorax treatment. The chest tube may include the one-way valve or the tube may simply be adapted for connection to a one-way valve or the like of a separate device.

Disposed on chest tube 10 is anchor member 15. Anchor member 15 comprises an inflatable member having an annular configuration. Anchor member 15 is disposed around chest tube 10 such that chest tube 10 passes through the orifice 16 of anchor member 15.

The anchor member 15 may comprise any suitable material capable of being filled with a fluid to expand its volume. Materials approved for medical use, such as those used in angioplasty balloons, are preferably used. Moreover, it is desirable for anchor member 15 to comprise a material and be configured to constrict the outer circumference of chest tube 10 in direct correlation to the increase in volume of anchor due to the amount of fill material being supplied to the anchor's interior 17.

Fill material 22 for inflating the anchor can be any suitable material. Preferably, the anchor is filled with a material non-toxic in the event of a rupture inside the patient's body. Presently preferred fill materials include fluids that can be delivered via a syringe, such as water or air. Air is a presently preferred fill material due to the fact that it does not add appreciable weight to the anchor.

A fill line 18 is associated with the anchor for aiding the provision of fluid to the interior 17 of the anchor 15. Fill line 18 may be of any suitable construction which provides for fluid communication from a fill port 19 to the anchor interior 22. Fill port 19 preferably comprises a port for receiving the tip 21 of syringe 20 carrying fill material 22. Fill port 19 preferably includes a spring valve 25 which allows communication to anchor 15 when actuated by tip 21 and which seals fluid-tight after retraction of tip 21 from fill port 19. Fill material 22 is deposited at fill port 19 until anchor member 15 is inflated to a volume suitable for holding a device, such as a chest tube 10, in a given orientation.

Fill port 19 may comprise a Luer-lock or like device for insuring a secure mating of the fill port and a syringe. In addition, fill port 19 may configured as a pressure balloon which serves as a proxy for determining the pressure of anchor 15 when anchor 15 is not visible to the user, e.g., when disposed inside the chest of a patient.

Anchor member 15 is preferably moveable along at least a length of the outside of chest tube 10. As will be appreciated, by being moveable, anchor member may be positioned inside the chest of a patient after an appropriate length of the chest tube 10 is inserted. Thereafter, anchor may be filled as described above to secure the chest tube 10 in its desired location without the need for sutures.

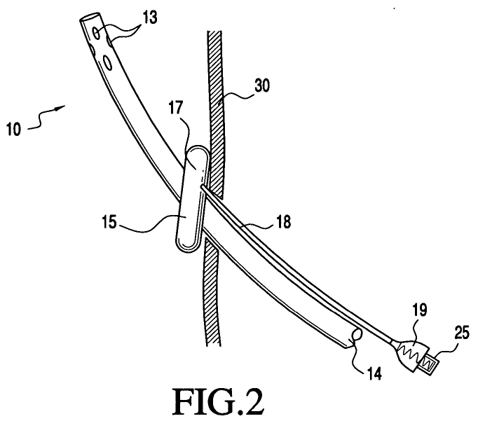

As depicted in FIG. 2, it is presently preferred that the annular anchor member 15 be disposed right inside the chest wall 30 and angled to be generally parallel to the chest wall 30.

In operation, the chest tube 10 embodiment of the present invention follows the convention steps for insertion previously described herein. However, according to the present invention, the anchor 15 is positioned along chest tube 10 in an area certain to be disposed inside the chest wall 30. Once the chest tube 10 is in its desired position (determined by listening to air movement using a stethoscope), the anchor 15 is positioned as described above by moving it along chest tube 10 to a position adjacent chest wall 30 by pull along fill line 18. The anchor 15 may then be filled with fill material 22 via fill line 18 while the tube 10 is connected to a one-way valve or underwater-seal apparatus (not shown) in order to clear air/blood from the pleural space. When it is desired to remove or reposition the chest tube 10, the anchor 15 may be deflated by aspiration with a syringe inserted into the fill port 19.

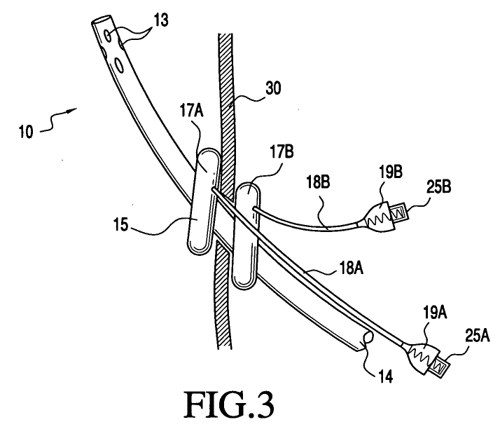

As shown in FIG. 3, an exterior anchor 17B may be used in combination (or to the exclusion of) the interior anchor 17A. According to this embodiment, each of the anchor bodies 17A, 17B have their own respective fill lines 18A, 18B and fill ports 19A, 19B. Such a construction allows for each anchor body to be positioned and inflated or deflated when desired. In addition, this construction allows for the anchor bodies to be used separately or together to anchor the chest tube depending on the circumstances. For example, it may be desirous to an exterior anchor in situations where a patient must be moved, suturing is unavailable or impractical, or the fear of the anchor slipping deeper into the chest cavity are present. However, in an alternative embodiment, anchor bodies 17A, 17B share a common fill line. With that configuration, the exterior anchor's size and pressure is a fair indication of the interior anchor's size and pressure.

In operation, a preferred method of using the dual anchor body embodiment is the same as that for the single anchor embodiment with the exception of positioning and inflating the exterior anchor either before or after the chest tube is secured to the Heimlich valve or evacuation device.

Those skilled in the art will appreciate that various adaptations and modifications of the above-described preferred embodiments can be configured without departing from the scope and spirit of the invention. Therefore, it is to be understood that, within the scope of the appended claims, the invention may be practiced other than as specifically described herein.

We claim:

1. A chest tube comprising:
an elongated tubular member having an insertion end and a non-insertion end disposed on opposite ends;
said insertion end defining a plurality of holes providing communication with an interior passageway through said tubular member;
said non-insertion end being adapted for facilitating the evacuating material from said insertion end through said passageway and out said non-insertion end;
an inflatable anchor disposed around an outer circumference of said tubular member;
means for inflating said anchor; whereby inflating said anchor grips said tubular member.

2. The chest tube of claim 1 wherein said means for inflating comprises a fill line in communication with said inflatable member.

3. The chest tube of claim 2 wherein said fill line is capable of receiving fill material from a syringe and depositing said material in an interior of said anchor, thereby inflating said anchor member and constricting movement of said tubular member.

4. The chest tube of claim 3 wherein said fill line includes a fill port comprising a spring-valve.

5. The chest tube of claim 3 wherein said fill material is selected from the group consisting of saline, water and air.

6. The chest tube of claim 5 further comprising a second inflatable anchor, said anchor disposed around an outer circumference of said tubular member; and
means for inflating said second inflatable anchor.

7. The chest tube of claim 7 wherein at least one of said inflatable anchors is attached to and moveable along an exterior surface of said tubular member.

8. A method of securing a chest tube in a desired position comprising:
inserting a chest tube in a desired position within a patient chest;
positioning an inflatable anchor defining an orifice disposed around a circumference of said chest tube adjacent and parallel to an interior side of a chest wall of said patient; and
inflating said anchor to a size suitable to restrict movement of said chest tube within said orifice.

9. The method of claim 8 further comprising:
positioning a second inflatable anchor having an orifice disposed around a circumference of said chest tube adjacent and parallel to an exterior side of said patient's chest wall; and
inflating said second anchor to a size suitable to constrict movement of said chest tube with said second anchor's orifice.

10. An anchor for securing a device comprising:
a first inflatable body and a second inflatable body, each of said bodies having an interior space adapted for holding a fill material and having a donut-shaped configuration; wherein an exterior surface of each of said bodies defines an orifice centrally therethrough; and
a first fill line in communication with the interior space of said first inflatable body and a second fill line in communication with the interior space of said second inflatable body; whereby inflating said bodies constricts a diameter of said orifice for preventing movement of a device passing therethrough.

11. The anchor of claim 10 wherein at least one of said inflatable bodies is selectively deflatable.

12. The anchor of claim 11 wherein each of said fill lines include a fill port having a one-way valve.

13. The anchor of claim 12 wherein said fill ports are adapted for receiving a syringe for adding or removing fill material from said interior space of its respective inflatable body.

14. The anchor of claim 13 wherein said fill material comprises a fluid.

15. The anchor of claim 10 wherein at least one of said inflatable bodies is movable along a length of said device in a deflated state and secured to said device in an inflated state.

16. The anchor of claim 15 wherein said device is a chest tube.

17. The anchor of claim 16 wherein said first inflatable body is positioned inside a patient's chest in the deflated state.

18. The anchor of claim 17 wherein the first inflatable body is positioned parallel and adjacent a chest wall of said patient.

19. The anchor of claim 18 wherein said first inflatable body is inflated with a fill material after being positioned.

20. The anchor of claim 19 wherein said second inflatable body is positioned and inflated on a side of said chest wall opposite said first inflatable body.
